(19) United States
(12) Patent Application Publication
(10) Pub. No.: US 2026/0061204 A1
(43) Pub. Date: Mar. 5, 2026

(54) ADHESIVE RECEPTACLE FOR MEDICAL IMPLANT

(71) Applicant: Cochlear Limited, Macquarie University, NSW (AU)

(72) Inventors: Floriaan Van Reusel, Mechelen (BE); Koen Erik Van den Heuvel, Mechelen (BE); Antonin Rambault, St. Aubin (FR)

(21) Appl. No.: 19/152,349

(22) PCT Filed: Feb. 20, 2024
(86) PCT No.: PCT/IB2024/051580

§ 371 Date: Jul. 30, 2025

Related U.S. Application Data
(60) Provisional application No. 63/489,649, filed on Mar. 10, 2023.

(51) Int. Cl. A61N 1/375

(52) U.S. Cl. CPC A61N 1/37518

(57) ABSTRACT

An apparatus includes a fixation portion configured to be affixed to a bone surface within a recipient's body. The apparatus further includes a mounting portion connected to the fixation portion, the mounting portion configured to hold a component within the recipient's body. The mounting portion includes a collar configured to extend at least partially around the component and a reservoir configured to receive an adhesive material within a predetermined volume at least partially bounded by an inner surface of the collar and an outer surface of the component.

### BACKGROUND

### Field

The present application relates generally to medical implants (e.g., implantable medical prostheses) having active components (e.g., transducers; actuators; microphones).

### Description of the Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect disclosed herein, an apparatus comprises a fixation portion configured to be affixed to a bone surface within a recipient's body. The apparatus further comprises a mounting portion connected to the fixation portion, the mounting portion configured to hold a component within the recipient's body. The mounting portion comprises a collar configured to extend at least partially around the component and a reservoir configured to receive an adhesive material within a predetermined volume at least partially bounded by an inner surface of the collar and an outer surface of the component.

In another aspect disclosed herein, an apparatus comprises a first component configured to be implanted within a recipient's body through a surgical hole. The apparatus further comprises a second component configured to be implanted within the recipient's body through the surgical hole. The second component has a first portion configured to be affixed to the recipient's body and a second portion configured to be affixed to the first component. The second portion comprises a receptacle configured to hold a predetermined amount of adhesive between the first component and the receptacle.

In another aspect disclosed herein, a method comprises affixing a fixation element to a location within a recipient's body, the fixation element configured to receive an assembly. The method further comprises inserting the assembly into the fixation element such that a position of the assembly can be adjusted relative to the fixation element. The method further comprises slidably adjusting the position of the assembly such that the assembly is at a location at which the assembly is configured to be operationally coupled to a target portion of the recipient's body. The method further comprises, upon the assembly being at the location, filling a volume between the fixation element and the assembly with adhesive.

### BRIEF DESCRIPTION OF THE DRAWINGS

Implementations are described herein in conjunction with the accompanying drawings, in which:

FIG. 1 is a perspective view of an example cochlear implant auditory prosthesis implanted in a recipient in accordance with certain implementations described herein;

FIG. 2 is a perspective view of an example fully implantable middle ear implant auditory prosthesis implanted in a recipient in accordance with certain implementations described herein;

FIG. 3 schematically illustrates an example apparatus in accordance with certain implementations described herein;

FIGS. 4A-4C schematically illustrate various views of an example fixation portion and an example mounting portion of the example apparatus of FIG. 3 in accordance with certain implementations described herein;

FIG. 5 schematically illustrates an example reservoir comprising a channel in accordance with certain implementations described herein;

FIGS. 6A-6D schematically illustrate various example positions of the component relative to the collar during implantation of the component in accordance with certain implementations described herein; and

FIG. 7 schematically illustrates an example method in accordance with certain implementations described herein.

### DETAILED DESCRIPTION

Certain implementations described herein provide a first component (e.g., transducer; hearing implant transducer) and a second component (e.g., fixation element) configured to be attached to a recipient's body and to the first component with an adjustable position of the first component relative to the second component (e.g., continuously adjustable; adjustable in a stepless manner). Once the first component is at a selected position, the first component is secured (e.g., affixed) to the second component by inserting an adhesive material (e.g., bone cement) into a reservoir (e.g., container; cup) that is in fluidic communication with surfaces of both the first component and the second component. The reservoir can have a predetermined volume that corresponds to a sufficient amount of adhesive material being used to achieve a satisfactory adhesive force (e.g., to provide confidence during the implantation that sufficient adhesive material has been used for a desired adhesive force strength and/or that excessive adhesive material has not been used which could extend the curing time), thereby facilitating consistency and usability during the surgical implantation process. The adhesive material can be inserted into the reservoir via an inlet that is positioned to facilitate the insertion of the adhesive material without disturbing the position of the first component (e.g., a tensionless insertion of the adhesive material).

The teachings detailed herein are applicable, in at least some implementations, to any type of implantable medical system utilizing an implantable transducer assembly configured to provide stimulation signals to a portion of the recipient's body in response to received information and/or control signals (e.g., implantable sensor prostheses; implantable stimulation system). For example, the implantable medical system can comprise an auditory prosthesis system configured to generate and apply stimulation signals that are perceived by the recipient as sounds (e.g., evoking a hearing percept). Such implantable transducer assemblies can include but are not limited to: electro-acoustic electrical/acoustic systems, cochlear implant devices, implantable hearing aid devices, middle ear implant devices, bone conduction devices (e.g., active bone conduction devices; passive bone conduction devices, percutaneous bone conduction devices; transcutaneous bone conduction devices), Direct Acoustic Cochlear Implant (DACI), middle ear transducer (MET), electro-acoustic implant devices, other types of auditory prosthesis devices (e.g., auditory brain stimulators), and/or combinations or variations thereof, or any other suitable hearing prosthesis system with or without one or more external components. Merely for ease of description, apparatus and methods disclosed herein are primarily described with reference to an illustrative auditory prosthesis system, namely a middle ear implant, but implementations can include any type of auditory prosthesis that can utilize the teachings detailed herein and/or variations thereof. Certain such implementations can be referred to as “partially implantable,” “semi-implantable,” “mostly implantable,” “fully implantable,” or “totally implantable” auditory prostheses.

The teachings detailed herein and/or variations thereof may also be used with a variety of other medical devices that provide a wide range of therapeutic benefits to recipients, patients, or other users. For example, other sensory prosthesis systems that are configured to evoke other types of neural or sensory (e.g., sight, tactile, smell, taste) percepts are compatible with certain implementations described herein, including but are not limited to: vestibular devices (e.g., vestibular implants), visual devices (e.g., bionic eyes), visual prostheses (e.g., retinal implants), somatosensory implants, and chemosensory implants. In some implementations, the teachings detailed herein and/or variations thereof can be utilized in other types of implantable medical devices beyond sensory prostheses. For example, apparatus and methods disclosed herein and/or variations thereof can be used with one or more of the following: sensors; cardiac pacemakers; drug delivery systems; defibrillators; functional electrical stimulation devices; catheters; brain implants; seizure devices (e.g., devices for monitoring and/or treating epileptic events); sleep apnea devices; electroporation; pain relief devices; etc. Implementations can include any type of medical system that can utilize the teachings detailed herein and/or variations thereof.

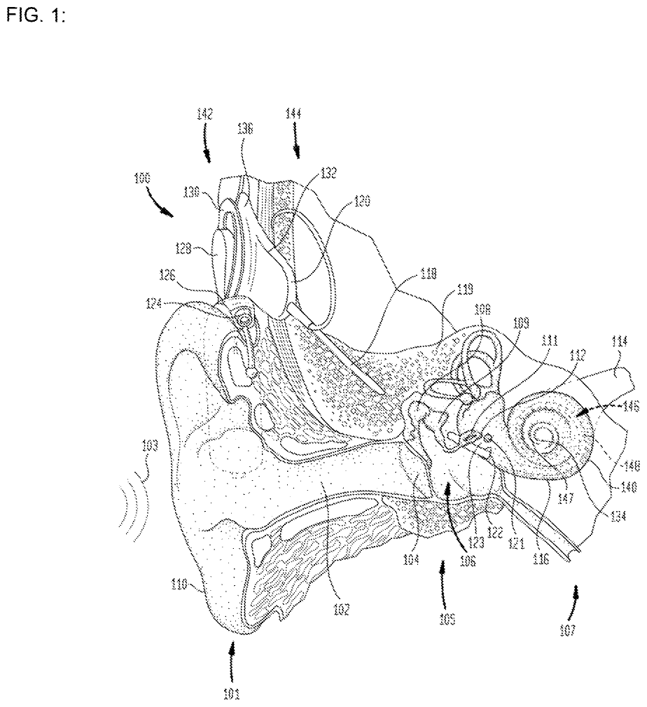

FIG. 1 is a perspective view of an example cochlear implant auditory prosthesis 100 implanted in a recipient in accordance with certain implementations described herein. The example auditory prosthesis 100 is shown in FIG. 1 as comprising an implanted stimulator unit 120 and a microphone assembly 124 that is external to the recipient (e.g., a partially implantable cochlear implant). An example auditory prosthesis 100 (e.g., a totally implantable cochlear implant; a mostly implantable cochlear implant) in accordance with certain implementations described herein can replace the external microphone assembly 124 shown in FIG. 1 with a subcutaneously implantable microphone assembly, as described more fully herein.

As shown in FIG. 1, the recipient has an outer ear 101, a middle ear 105, and an inner ear 107. In a fully functional ear, the outer ear 101 comprises an auricle 110 and an ear canal 102. An acoustic pressure or sound wave 103 is collected by the auricle 110 and is channeled into and through the ear canal 102. Disposed across the distal end of the ear canal 102 is a tympanic membrane 104 which vibrates in response to the sound wave 103. This vibration is coupled to oval window or fenestra ovalis 112 through three bones of middle ear 105, collectively referred to as the ossicles 106 and comprising the malleus 108, the incus 109, and the stapes 111. The bones 108, 109, and 111 of the middle ear 105 serve to filter and amplify the sound wave 103, causing the oval window 112 to articulate, or vibrate in response to vibration of the tympanic membrane 104. This vibration sets up waves of fluid motion of the perilymph within cochlea 140. Such fluid motion, in turn, activates tiny hair cells (not shown) inside the cochlea 140. Activation of the hair cells causes appropriate nerve impulses to be generated and transferred through the spiral ganglion cells (not shown) and auditory nerve 114 to the brain (also not shown) where they are perceived as sound.

As shown in FIG. 1, the example auditory prosthesis 100 comprises one or more components which are temporarily or permanently implanted in the recipient. The example auditory prosthesis 100 is shown in FIG. 1 with an external component 142 which is directly or indirectly attached to the recipient's body, and an internal component 144 which is temporarily or permanently implanted in the recipient (e.g., positioned in a recess of the temporal bone adjacent auricle 110 of the recipient). The external component 142 typically comprises one or more sound input elements (e.g., an external microphone 124) for detecting sound, a sound processing unit 126 (e.g., disposed in a Behind-The-Ear unit), a power source (not shown), and an external transmitter unit 128. In the illustrative implementations of FIG. 1, the external transmitter unit 128 comprises an external coil 130 (e.g., a wire antenna coil comprising multiple turns of electrically insulated single-strand or multi-strand platinum or gold wire) and, preferably, a magnet (not shown) secured directly or indirectly to the external coil 130. The external coil 130 of the external transmitter unit 128 is part of an inductive radio frequency (RF) communication link with the internal component 144. The sound processing unit 126 processes the output of the microphone 124 that is positioned externally to the recipient's body, in the depicted implementation, by the recipient's auricle 110. The sound processing unit 126 processes the output of the microphone 124 and generates encoded signals, sometimes referred to herein as encoded data signals, which are provided to the external transmitter unit 128 (e.g., via a cable). As will be appreciated, the sound processing unit 126 can utilize digital processing techniques to provide frequency shaping, amplification, compression, and other signal conditioning, including conditioning based on recipient-specific fitting parameters.

The power source of the external component 142 is configured to provide power to the auditory prosthesis 100, where the auditory prosthesis 100 includes a battery (e.g., located in the internal component 144, or disposed in a separate implanted location) that is recharged by the power provided from the external component 142 (e.g., via a transcutaneous energy transfer link). The transcutaneous energy transfer link is used to transfer power and/or data to the internal component 144 of the auditory prosthesis 100. Various types of energy transfer, such as infrared (IR), electromagnetic, capacitive, and inductive transfer, may be used to transfer the power and/or data from the external component 142 to the internal component 144. During operation of the auditory prosthesis 100, the power stored by the rechargeable battery is distributed to the various other implanted components as needed.

The internal component 144 comprises an internal receiver unit 132, a stimulator unit 120, and an elongate electrode assembly 118. In some implementations, the internal receiver unit 132 and the stimulator unit 120 are hermetically sealed within a biocompatible housing. The internal receiver unit 132 comprises an internal coil 136 (e.g., a wire antenna coil comprising multiple turns of electrically insulated single-strand or multi-strand platinum or gold wire), and preferably, a magnet (also not shown) fixed relative to the internal coil 136. The internal receiver unit 132 and the stimulator unit 120 are hermetically sealed within a biocompatible housing, sometimes collectively referred to as a stimulator/receiver unit. The internal coil 136 receives power and/or data signals from the external coil 130 via a transcutaneous energy transfer link (e.g., an inductive RF link). The stimulator unit 120 generates electrical stimulation signals based on the data signals, and the stimulation signals are delivered to the recipient via the elongate electrode assembly 118.

The elongate electrode assembly 118 has a proximal end connected to the stimulator unit 120, and a distal end implanted in the cochlea 140. The electrode assembly 118 extends from the stimulator unit 120 to the cochlea 140 through the mastoid bone 119. In some implementations, the electrode assembly 118 may be implanted at least in the basal region 116, and sometimes further. For example, the electrode assembly 118 may extend towards apical end of cochlea 140, referred to as cochlea apex 134. In certain circumstances, the electrode assembly 118 may be inserted into the cochlea 140 via a cochleostomy 122. In other circumstances, a cochleostomy may be formed through the round window 121, the oval window 112, the promontory 123, or through an apical turn 147 of the cochlea 140.

The elongate electrode assembly 118 comprises a longitudinally aligned and distally extending array 146 of electrodes or contacts 148, sometimes referred to as electrode or contact array 146 herein, disposed along a length thereof. Although the electrode array 146 can be disposed on the electrode assembly 118, in most practical applications, the electrode array 146 is integrated into the electrode assembly 118 (e.g., the electrode array 146 is disposed in the electrode assembly 118). As noted, the stimulator unit 120 generates stimulation signals which are applied by the electrodes 148 to the cochlea 140, thereby stimulating the auditory nerve 114.

While FIG. 1 schematically illustrates an auditory prosthesis 100 utilizing an external component 142 comprising an external microphone 124, an external sound processing unit 126, and an external power source, in certain other implementations, one or more of the microphone 124, sound processing unit 126, and power source are implantable on or within the recipient (e.g., within the internal component 144). For example, the auditory prosthesis 100 can have each of the microphone 124, sound processing unit 126, and power source implantable on or within the recipient (e.g., encapsulated within a biocompatible assembly located subcutaneously), and can be referred to as a totally implantable cochlear implant (“TICI”). For another example, the auditory prosthesis 100 can have most components of the cochlear implant (e.g., excluding the microphone, which can be an in-the-ear-canal microphone) implantable on or within the recipient, and can be referred to as a mostly implantable cochlear implant (“MICI”).

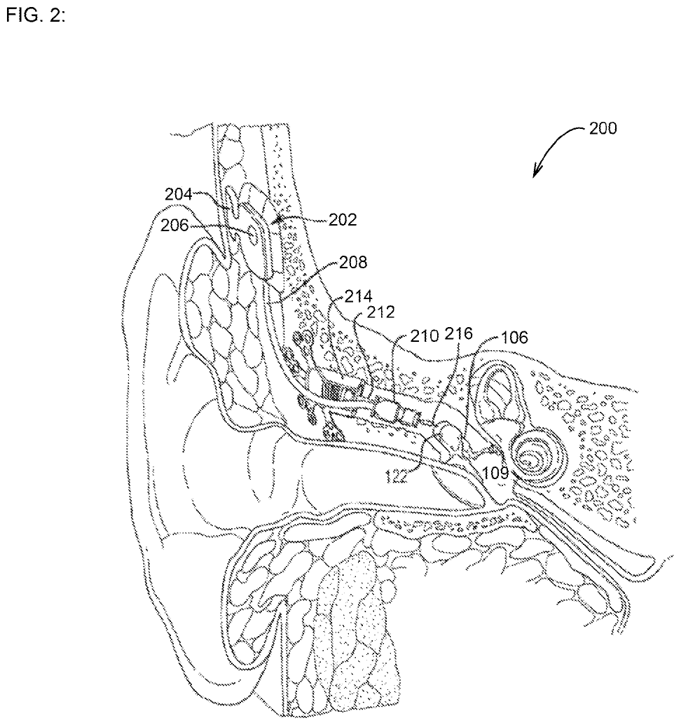

FIG. 2 schematically illustrates a perspective view of an example fully implantable auditory prosthesis 200 (e.g., fully implantable middle ear implant or totally implantable acoustic system), implanted in a recipient, utilizing an acoustic actuator in accordance with certain implementations described herein. The example auditory prosthesis 200 of FIG. 2 comprises a biocompatible implantable assembly 202 (e.g., comprising an implantable capsule) located subcutaneously (e.g., beneath the recipient's skin and on a recipient's skull). While FIG. 2 schematically illustrates an example implantable assembly 202 comprising a microphone, in other example auditory prostheses 200, a pendant microphone can be used (e.g., connected to the implantable assembly 202 by a cable). The implantable assembly 202 includes a signal receiver 204 (e.g., comprising a coil element) and an acoustic transducer (e.g., a microphone assembly 206 comprising a diaphragm and an electret or piezoelectric transducer) that is positioned to receive acoustic signals through the recipient's overlying tissue. The implantable assembly 202 may further be utilized to house a number of components of the fully implantable auditory prosthesis 200. For example, the implantable assembly 202 can include an energy storage device and a signal processor (e.g., a sound processing unit). Various additional processing logic and/or circuitry components can also be included in the implantable assembly 202 as a matter of design choice.

For the example auditory prosthesis 200 shown in FIG. 2, the signal processor of the implantable assembly 202 is in operative communication (e.g., electrically interconnected via a wire 208) with an actuator 210 (e.g., comprising a transducer configured to generate mechanical vibrations in response to electrical signals from the signal processor). Examples of actuators 210 compatible with certain implementations described herein include, but are not limited to: piezoelectric stack, piezoelectric disk; microelectromechanical system (MEMS)-based activator. In certain implementations, the example auditory prosthesis 100, 200 shown in FIGS. 1 and 2 can comprise an implantable microphone assembly, such as the microphone assembly 206 shown in FIG. 2. For such an example auditory prosthesis 100, the signal processor of the implantable assembly 202 can be in operative communication (e.g., electrically interconnected via a wire) with the microphone assembly 206 and the stimulator unit of the main implantable component (e.g., the stimulator unit 120). In certain implementations, at least one of the microphone assembly 206 and the signal processor (e.g., a sound processing unit) is implanted on or within the recipient.

The actuator 210 of the example auditory prosthesis 200 shown in FIG. 2 is supportably connected to a positioning system 212, which in turn, is connected to a bone anchor 214 mounted within the recipient's mastoid process (e.g., via a hole drilled through the skull). The actuator 210 includes a connection apparatus 216 for connecting the actuator 210 to the ossicles 106 of the recipient. In a connected state, the connection apparatus 216 provides a communication path for acoustic stimulation of the ossicles 106 (e.g., through transmission of vibrations from the actuator 210 to the incus 109).

During normal operation, ambient acoustic signals (e.g., ambient sound) impinge on the recipient's tissue and are received transcutaneously at the microphone assembly 206. Upon receipt of the transcutaneous signals, a signal processor within the implantable assembly 202 processes the signals to provide a processed audio drive signal via wire 208 to the actuator 210. As will be appreciated, the signal processor may utilize digital processing techniques to provide frequency shaping, amplification, compression, and other signal conditioning, including conditioning based on recipient-specific fitting parameters. The audio drive signal causes the actuator 210 to transmit vibrations at acoustic frequencies to the connection apparatus 216 to affect the desired sound sensation via mechanical stimulation of the incus 109 of the recipient.

The subcutaneously implantable microphone assembly 202 is configured to respond to auditory signals (e.g., sound; pressure variations in an audible frequency range) by generating output signals (e.g., electrical signals; optical signals; electromagnetic signals) indicative of the auditory signals received by the microphone assembly 202, and these output signals are used by the auditory prosthesis 100, 200 to generate stimulation signals which are provided to the recipient's auditory system. To compensate for the decreased acoustic signal strength reaching the microphone assembly 202 by virtue of being implanted, the diaphragm of an implantable microphone assembly 202 can be configured to provide higher sensitivity than are external non-implantable microphone assemblies. For example, the diaphragm of an implantable microphone assembly 202 can be configured to be more robust and/or larger than diaphragms for external non-implantable microphone assemblies.

The example auditory prostheses 100 shown in FIG. 1 utilizes an external microphone 124 and the auditory prosthesis 200 shown in FIG. 2 utilizes an implantable microphone assembly 206 comprising a subcutaneously implantable acoustic transducer. In certain implementations described herein, the auditory prosthesis 100 utilizes one or more implanted microphone assemblies on or within the recipient. In certain implementations described herein, the auditory prosthesis 200 utilizes one or more microphone assemblies that are positioned external to the recipient and/or that are implanted on or within the recipient, and utilizes one or more acoustic transducers (e.g., actuator 210) that are implanted on or within the recipient. In certain implementations, an external microphone assembly can be used to supplement an implantable microphone assembly of the auditory prosthesis 100, 200. Thus, the teachings detailed herein and/or variations thereof can be utilized with any type of external or implantable microphone arrangement, and the acoustic transducers shown in FIGS. 1 and 2 are merely illustrative.

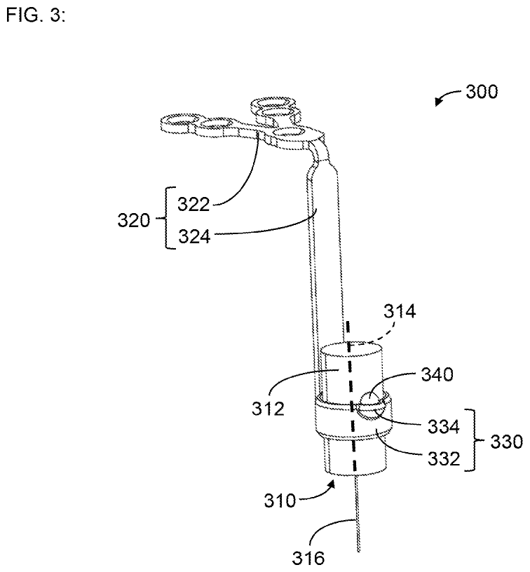

FIG. 3 schematically illustrates an example apparatus 300 in accordance with certain implementations described herein. The apparatus 300 can be configured to affix a component 310 (e.g., a portion of a medical device; a transducer) within a recipient's body. The apparatus 300 comprises a fixation portion 320 configured to be affixed to a bone surface within the recipient's body. The apparatus 300 further comprises a mounting portion 330 connected to the fixation portion 320. The mounting portion 330 is configured to hold the component 310 within the recipient's body. The mounting portion 330 comprises a collar 332 configured to extend at least partially around the component 310 and a reservoir 334 configured to receive an adhesive material 340 within a predetermined volume at least partially bounded by an inner surface 336 of the collar 332 and an outer surface 312 of the component 310.

In certain implementations, the apparatus 300 comprises an auditory prosthesis system comprising a middle ear assembly that is based on two-point fixation with one fixation point at a fixation position (e.g., a surface of the recipient's skull 303) and a second fixation point at the target portion of the recipient's body (e.g., a middle ear target; ossicle 106; incus 109; tympanic membrane 104; oval window 112; round window 121; bone surrounding a cochlea; promontory 123; horizontal, posterior, or superior semicircular canals), with the middle ear assembly bridging the physical gap between the two fixation points. In certain other implementations, the middle ear assembly is only connected to the target portion of the recipient's body (e.g., the middle ear assembly is floating; only affixed to the tympanic membrane 104 or the ossicles 106). Other types of implantable medical devices besides auditory prosthesis systems are also compatible with certain implementations described herein.

In certain implementations, the component 310 comprises an active component (e.g., transducer; actuator; microphone; optical sensor; magnetic induction sensor) configured to be in operative communication with a target portion of the recipient's body (e.g., an ossicle 106, a portion of a cochlea 140, a portion of the otic capsule, or a semicircular canal of the recipient's body). For example, the component 310 can comprise an acoustic actuator (e.g., actuator 210) configured to generate mechanical vibrations at acoustic frequencies in response to electrical signals from a signal processor and to transmit the vibrations (e.g., via an elongate member 316) to the ossicles 106 of the recipient to create a hearing percept. For another example, the component 310 can comprise a microphone (e.g., vibration sensor; electret microphone; electromechanical microphone, piezoelectric microphone; MEMS microphone; accelerometer; optical interferometer; pressure sensor) configured to sense or detect mechanical vibrations from a portion of the recipient's body (e.g., the ossicles 106) and to generate electrical and/or optical signals in response. The electrical signals can be provided to a sound processing unit and/or stimulation device configured to respond to the electrical signals by generating stimulation signals provided to the recipient to create a hearing percept.

In certain implementations, the outer surface 312 of the component 310 has a substantially circular cross-sectional shape in a plane perpendicular to a longitudinal axis 314 of the component 310 (e.g., the outer surface 312 is substantially circularly symmetric about the longitudinal axis 314), while in certain other implementations, the outer surface 312 has other cross-sectional shapes (e.g., oval; square; rectangular; irregular) in a plane perpendicular to the longitudinal axis 314.

In certain implementations, the active component of the component 310 is in mechanical communication with the target portion of the recipient's body via an elongate member 316 (e.g., rod, cable, wire, tube) comprising a biocompatible material (e.g., titanium; titanium alloy; platinum; gold; stainless steel; nitinol; silicone; plastic; ceramic) and extending from an end portion of the component 310 to the target portion of the recipient's body (e.g., extending a length in a range of 5 millimeters to 40 millimeters; in a range of 5 millimeters to 15 millimeters). The elongate member 316 (e.g., rigid; flexible; straight; curved) can have a first end portion mechanically coupled to the component 310 and a second end portion be configured to be in mechanical communication with the target portion of the recipient's body such that mechanical vibrations propagate along the elongate member 316 between the component 310 and the target portion of the recipient's body. The mechanical coupling between the elongate member 316 and the target portion of the recipient's body can be accomplished in various ways (e.g., a surface-to-surface mechanical contact; affixed directly with bone cement or another type of biocompatible adhesive; a clip configured to be slid onto the target portion).

In certain other implementations, the active component of the component 310 comprises a sensor (e.g., optical sensor; magnetic induction sensor) facing towards the target portion of the recipient's body and configured to sense or detect motion (e.g., vibrations) of the target portion of the recipient's body (e.g., without an elongate member 316; without contacting the target portion). While certain implementations are described herein as comprising a component 310 comprising a transducer, in certain other implementations, the apparatus 300 comprises a different component (e.g., reservoir; valve; pump) configured to be in operative communication with the target portion of the recipient's body via an elongate member 316 comprising a fluid conduit (e.g., tube) configured to provide at least one liquid medicament to the target portion of the recipient's body.

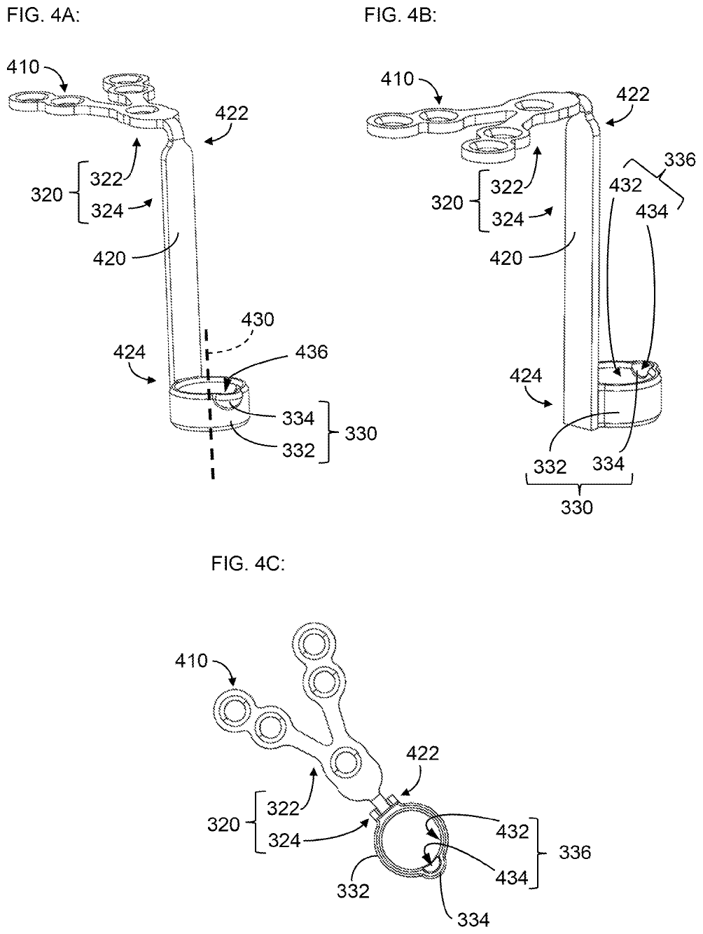

FIGS. 4A-4C schematically illustrate various views of an example fixation portion 320 and an example mounting portion 330 of the example apparatus 300 of FIG. 3 (without the adhesive material 340 and without having the component 310 held by the mounting portion 330) in accordance with certain implementations described herein. FIG. 4A schematically illustrates a front, first side, and top perspective view, FIG. 4B schematically illustrates a rear, first side, and top perspective view, and FIG. 4C schematically illustrates a top view.

In certain implementations, the fixation portion 320 (e.g., bone anchor 214) comprises a first portion 322 (e.g., bracket) comprising a biocompatible material (e.g., titanium; titanium alloy; plastic; ceramic). The first portion 322 can be configured to be affixed to a first location on and/or within the recipient's body (e.g., beneath the recipient's skin and on a recipient's skull; on a machined surface of the skull bone; on an outer bone surface; on a top bone surface). For example, as shown in FIGS. 4A-4C, the first portion 322 can comprise one or more holes 410 or other structures configured to be held onto the first location by a screw, suture, adhesive, or other fixation device (not shown) and/or osseointegration. In certain implementations, the first portion 322 can comprise a mechanism (e.g., z-adjustment microdrive and compression unit) configured to controllably adjust a linear position (e.g., depth) of the mounting portion 330 (e.g., about 4 to 10 millimeters) and/or an angle of the mounting portion 330 relative to a target portion of the recipient's body.

The fixation portion 320 can further comprise a second portion 324 comprising a biocompatible material (e.g., titanium; titanium alloy; plastic; ceramic). The second portion 324 can be configured to extend from the first portion 322 towards a target portion of the recipient's body (e.g., through a hole drilled through the skull; in an inner ear region; in a middle ear region; within a cochleovestibular region; to an ossicle 106; to a cochlea 140) to be at least partially within a region (e.g., middle ear region; mastoid bone cavity; channel; cavity; naturally-occurring; extending through the skull bone; drilled or otherwise formed through surgical techniques). At least one of the first portion 322 and the second portion 324 can be configured to be plastically deformed (e.g., bent and/or twisted; controlled by a practitioner during implantation of the apparatus 300) such that the position (e.g., linear position; depth) and/or orientation of the mounting portion 330 is adjusted relative to the target portion of the recipient's body. For example, the second portion 324 can comprise at least one plastically deformable arm 420 having a first end portion 422 affixed to the first portion 322 and a second end portion 424 affixed to the collar 332. In certain implementations, the first portion 322 and the second portion 324 are integral with one another, while in certain other implementations, the first portion 322 and the second portion 324 are separate components that are affixed to one another. While the second portion 324 is described herein as being part of the fixation portion 320, in certain other implementations, the second portion 324 is part of the mounting portion 330.

In certain implementations, the collar 332 of the mounting portion 330 comprises a biocompatible material (e.g., titanium; titanium alloy; plastic; ceramic) and is configured to hold the component 310. In certain implementations, the collar 332 and the second portion 324 are integral with one another, while in certain other implementations, the collar 332 and the second portion 324 are separate components that are affixed to one another. The mounting portion 330 can be configured to be adjusted (e.g., during implantation of the apparatus 300) to modify a position (e.g., linear position; depth) and/or orientation of the collar 332 relative to the second portion 324 and/or the target portion of the recipient's body. For example, the collar 332 can be adjustably affixed to the second end portion 424 of the arm 420 of the second portion 324 (e.g., by a mechanism configured to be adjusted by the practitioner during or prior to implantation). Once mounted within the collar 332, the component 310 can be in mechanical communication with the fixation portion 320 and in operative communication with the target portion of the recipient's body (e.g., an ossicle 106; incus 109) spaced from the fixation portion 320.

In certain implementations, the collar 332 is configured to extend along and at least partially around the outer surface 312 of the component 310. For example, the inner surface 336 of the collar 332 can extend completely around the outer surface 312 of the component 310 (e.g., around a substantially cylindrical portion of the outer surface 312) or can extend only partly around the outer surface 312 (e.g., the collar 332 comprising at least two sections with a gap therebetween). Both the inner surface 336 of the collar 332 and the outer surface 312 of the component 310 can have substantially the same cross-sectional shape and can be configured to mate with one another (e.g., with the longitudinal axis 314 of the component 310 coincident or substantially parallel to the longitudinal axis 430 of the collar 332). For example, as schematically illustrated by FIG. 3, the cross-sectional shape can be substantially circular (e.g., the collar 332 comprising a ring with the inner surface 336 substantially circularly symmetric about the longitudinal axis 430 of the collar 332). In certain other implementations, the inner surface 336 has other cross-sectional shapes (e.g., oval; square; rectangular; irregular) in a plane perpendicular to the longitudinal axis 430 of the collar 332. In certain other implementations, the component 310 and the collar 332 form a ball joint (e.g., the component 310 comprising a ball portion and the collar 332 comprising a socket portion configured to receive the ball portion such that the component 310 can be rotated within the collar 332 to adjust an orientation of the component 310 relative to the recipient's body).

In certain implementations, the adhesive material 340 comprises a biocompatible adhesive (e.g., bone cement) configured to be in a liquid phase during implantation (e.g., during insertion into the reservoir 334) and a solid phase subsequent to implantation (e.g., after curing of the adhesive material). For example, the adhesive material 340 can selected from the group consisting of: silicone; synthetic rubber; epoxy (e.g., one-part; two-part); epoxy-polyurethane mixture; acrylate; cyanoacrylate; light-cured materials.

In certain implementations, the reservoir 334 (e.g., receptacle; cup; container) comprises a predetermined volume between the inner surface 336 of the collar 332 and the outer surface 312 of the component 310 (e.g., the predetermined volume at least partially bounded by the second portion 434 of the inner surface 336 and the outer surface 312 of the component 310). The predetermined volume is configured to contain a predetermined amount of the adhesive material 340 (e.g., an amount expected to be sufficient for affixing the component 310 in position within the collar 332). As schematically illustrated by FIGS. 3 and 4A-4C, the shape of the reservoir 334 can be generally the shape of a quarter of a sphere, although other shapes are also compatible with certain implementations described herein. While FIGS. 3 and 4A-4C show a single reservoir 334, the mounting portion 330 can comprise multiple reservoirs 334 (e.g., positioned at different locations around the component 310).

In certain implementations, the reservoir 334 is configured to be filled with the adhesive material 340 by the practitioner without substantially changing the position of the component 310 relative to the collar 332 (e.g., without substantially disturbing the coupling of the elongate member 316 with the target portion of the recipient's body; tensionless). For example, the reservoir 334 can comprise an inlet 436 (e.g., opening) configured to receive the adhesive material 340 with the component 310 positioned within the collar 332 and the fixation portion 320 affixed to the bone surface. As schematically illustrated by FIGS. 3 and 4A-4C, the inlet 436 can be positioned at a top edge of the collar 332 (e.g., an edge closest to the first portion 322 of the fixation portion 320) and at a location spaced away from the second portion 324 of the fixation portion 320 (e.g., at an opposite side of the collar 332 from the second portion 324), such that the inlet 436 is accessible by the practitioner (e.g., along a direction substantially parallel to the longitudinal axis 314; extending through a naturally-occurring hole or a hole drilled or otherwise formed using surgical techniques; extending through a bone surface, such as the skull, into an inner ear region, a middle ear region, or a cochleovestibular region). Other positions for the inlet 436 are also compatible with certain implementations described herein.

In certain implementations, one or both of the outer surface 312 of the component 310 and the inner surface 336 of the collar 332 comprises at least one concave portion (e.g., recess) configured to receive and hold the predetermined amount of the adhesive material 340. For example, as schematically illustrated by FIGS. 4A-4C, the inner surface 336 of the collar 332 can comprise a substantially cylindrical first portion 432 and a second portion 434 that extends radially outwardly (e.g., away from the longitudinal axis 430) further than does the substantially cylindrical first portion 432. The outer surface 312 of the component 310 can have an indentation that is configured to be filled by the adhesive material 340.

In certain implementations, the reservoir 334 comprises a fill line or other guide element configured to indicate to the practitioner that the predetermined volume contains the predetermined amount of adhesive material 340 when the adhesive material 340 reaches the fill line. For example, as shown in FIG. 3, the guide element can comprise an outer lip of the inlet 436 and the predetermined volume can contain the predetermined amount of adhesive material 340 when the adhesive material 340 fills the reservoir 334 to the outer lip and has a surface tension boundary that extends beyond the inlet 436.

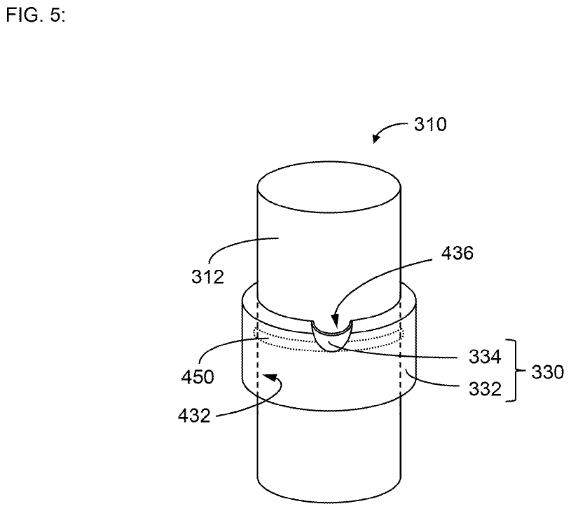

In certain implementations, the reservoir 334 comprises at least one concave channel 450 extending along the inner surface 336 of the collar 332, the channel 450 bounded by the inner surface 336 of the collar 332 and the outer surface 312 of the component 310. FIG. 5 schematically illustrates an example reservoir 334 comprising a channel 450 in accordance with certain implementations described herein. The channel 450 extends at least partially around the component 310 (e.g., completely encircling the component 310; partially encircling the component 310) and is configured to receive the adhesive material 340 from the cup-shaped portion of the reservoir 334 (e.g., the adhesive material 340 wicks into the channel 450 from the reservoir 334). In certain implementations, a generally annular region (e.g., gap) between the first portion 432 of the inner surface 336 and the outer surface 312 of the component 310 is in fluidic communication with the reservoir 334 such that the adhesive material 340 wicks into the region from the reservoir 334.

In certain implementations, the collar 332 is configured to allow the component 310 to be adjustably moved (e.g., controllably slid) within the collar 332 along the longitudinal axis 314 of the component 310 prior to introduction of the adhesive material 340 within the reservoir 334 and to be fixed at (e.g., unmovable from) a position relative to the collar 332 after the adhesive material 340 has been placed within the reservoir 334. In certain implementations, the component 310 is continuously movable within the collar 332 (e.g., movable in a stepless manner), while in certain other implementations, the component 310 has a set of discrete positions relative to the collar 332 (e.g., movable in a stepped manner).

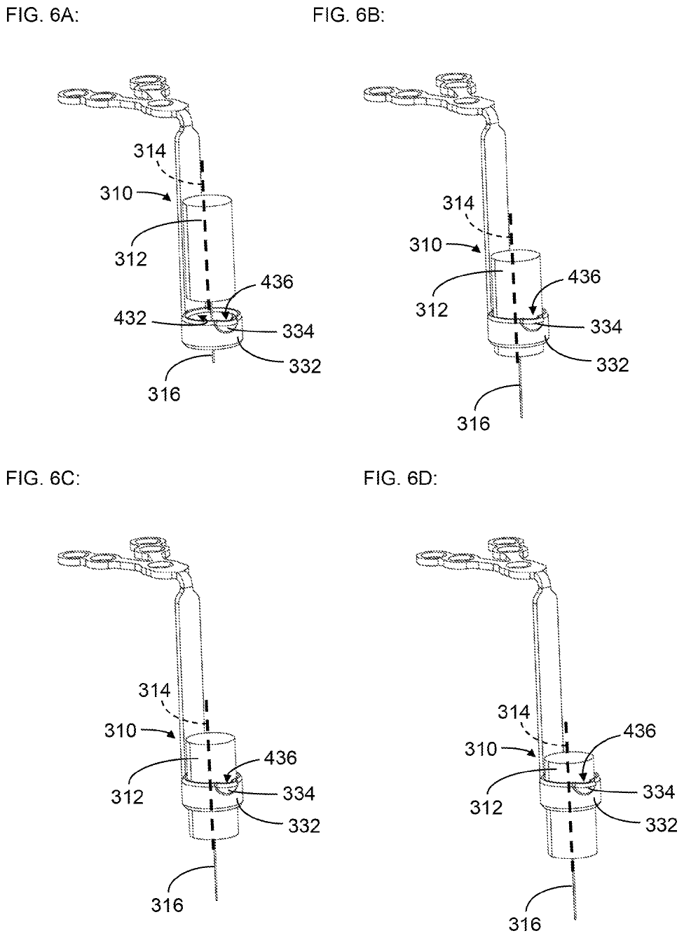

FIGS. 6A-6D schematically illustrate various example positions of the component 310 relative to the collar 332 during implantation of the component 310 in accordance with certain implementations described herein. The collar 332 can be configured to receive the component 310 with the outer surface 312 of the component 310 in adjustably slidable contact with the inner surface 336 of the collar 332. As shown in FIG. 6A, the component 310 can initially be positioned with the elongate member 316 of the component 310 inserted into the collar 332. As shown in FIGS. 6B-6D, the outer surface 312 of the component 310 can be inserted into the collar 332 and moved (e.g., slid) to adjust the distance of the component 310 relative to the target portion of the recipient's body. Upon the component 310 being placed at a selected position (e.g., with the elongate member 316 in mechanical communication with the target portion of the recipient's body), the adhesive material 340 can be placed within the reservoir 334 via the inlet 436 (see, e.g., FIG. 3). For example, the adhesive material 340 can be contained within a syringe that is used to place drops of the adhesive material 340 into the reservoir 334 to fill the predetermined volume.

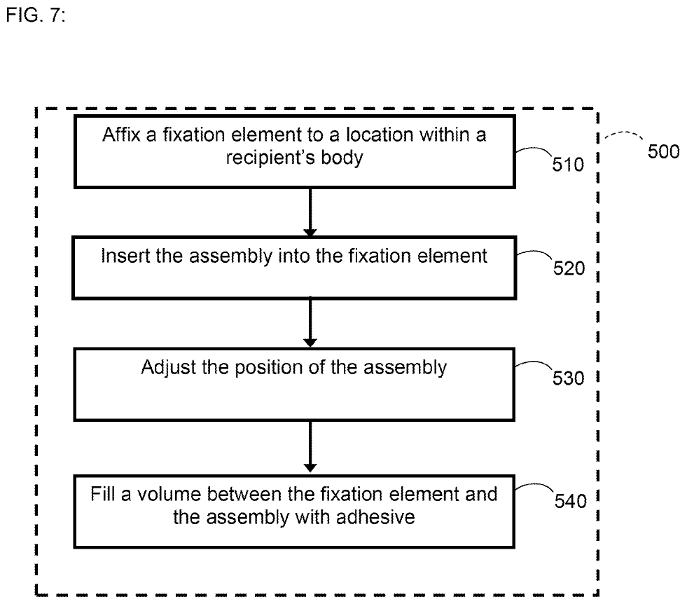

FIG. 7 is a flow diagram of an example method 500 (e.g., implantation process) in accordance with certain implementations described herein. While the example method 500 is described herein by referring to the example apparatus 300 of FIGS. 3, 4A-4C, 5, and 6A-6D, other apparatuses are also compatible with the example method 500 in accordance with certain implementations described herein. For example, the method 500 described herein can be applied to any of a variety of implantable medical devices.

In an operational block 510, the method 500 comprises affixing a fixation element (e.g., comprising fixation portion 320 and mounting portion 330) to a location within a recipient's body. The fixation element is configured to receive an assembly (e.g., component 310). For example, the assembly can be affixed to a bone surface portion (e.g., beneath the recipient's skin and on a recipient's skull; on a machined surface of the skull bone; on an outer bone surface; on a top bone surface).

In an operational block 520, the method 500 further comprises inserting the assembly into the fixation element such that a position of the assembly can be adjusted (e.g., slidably adjusted) relative to the fixation element. In an operational block 530, the method 500 further comprises adjusting the position of the assembly such that the assembly is at a location at which the assembly is configured to be operationally coupled to a target portion of the recipient's body. For example, the assembly can be held by a tool (e.g., tweezers; not shown) being manipulated by a practitioner during the method 500, by which the tool holds and inserts the assembly into the fixation element and slides the assembly within the fixation element until the assembly is at a selected position.

In an operational block 540, the method 500 further comprises, upon the assembly being at the location, filling a volume between the fixation element and the assembly with adhesive. The volume can be between an inner surface of the fixation element and an outer surface of the assembly. In certain implementations in which the fixation element comprises a guide element (e.g., fill line) indicative of the volume containing a predetermined amount of the adhesive (e.g., an amount sufficient for proper adhesion force between the fixation element and the assembly), filling the volume comprises stopping said filling when the adhesive reaches the guide element.

In certain implementations, the method 500 further comprises attaching the assembly to the target portion of the recipient's body. For example, the elongate member 316 of the component 310 can be attached to the target portion of the recipient's body prior to said filling the volume with adhesive, or after said filling the volume with adhesive.

Although commonly used terms are used to describe the systems and methods of certain implementations for ease of understanding, these terms are used herein to have their broadest reasonable interpretations. Although various aspects of the disclosure are described with regard to illustrative examples and implementations, the disclosed examples and implementations should not be construed as limiting. Conditional language, such as, among others, “can,” “could,” “might,” or “may,” unless specifically stated otherwise, or otherwise understood within the context as used, is generally intended to convey that certain implementations include, while other implementations do not include, certain features, elements and/or steps. Thus, such conditional language is not generally intended to imply that features, elements and/or steps are in any way required for one or more implementations or that one or more implementations necessarily include logic for deciding, with or without user input or prompting, whether these features, elements and/or steps are included or are to be performed in any particular implementation. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

It is to be appreciated that the implementations disclosed herein are not mutually exclusive and may be combined with one another in various arrangements. In addition, although the disclosed methods and apparatuses have largely been described in the context of conventional cochlear implants, various implementations described herein can be incorporated in a variety of other suitable devices, methods, and contexts. More generally, as can be appreciated, certain implementations described herein can be used in a variety of implantable medical device contexts.

Language of degree, as used herein, such as the terms “approximately,” “about,” “generally,” and “substantially,” represent a value, amount, or characteristic close to the stated value, amount, or characteristic that still performs a desired function or achieves a desired result. For example, the terms “approximately,” “about,” “generally,” and “substantially” may refer to an amount that is within ±10% of, within ±5% of, within ±2% of, within ±1% of, or within ±0.1% of the stated amount. As another example, the terms “generally parallel” and “substantially parallel” refer to a value, amount, or characteristic that departs from exactly parallel by +10 degrees, by +5 degrees, by ±2 degrees, by ±1 degree, or by +0.1 degree, and the terms “generally perpendicular” and “substantially perpendicular” refer to a value, amount, or characteristic that departs from exactly perpendicular by ±10 degrees, by ±5 degrees, by ±2 degrees, by ±1 degree, or by ±0.1 degree. The ranges disclosed herein also encompass any and all overlap, sub-ranges, and combinations thereof. Language such as “up to,” “at least,” “greater than,” less than,” “between,” and the like includes the number recited. As used herein, the meaning of “a,” “an,” and “said” includes plural reference unless the context clearly dictates otherwise. Also, as used in the description herein, the meaning of “in” includes “into” and “on,” unless the context clearly dictates otherwise.

While the methods and systems are discussed herein in terms of elements labeled by ordinal adjectives (e.g., first, second, etc.), the ordinal adjective are used merely as labels to distinguish one element from another (e.g., one signal from another or one circuit from one another), and the ordinal adjective is not used to denote an order of these elements or of their use.

The invention described and claimed herein is not to be limited in scope by the specific example implementations herein disclosed, since these implementations are intended as illustrations, and not limitations, of several aspects of the invention. Any equivalent implementations are intended to be within the scope of this invention. Indeed, various modifications of the invention in form and detail, in addition to those shown and described herein, will become apparent to those skilled in the art from the foregoing description. Such modifications are also intended to fall within the scope of the claims. The breadth and scope of the invention should not be limited by any of the example implementations disclosed herein, but should be defined only in accordance with the claims and their equivalents.

1. An apparatus comprising:
a fixation portion configured to be affixed to a bone surface within a recipient's body; and
a mounting portion connected to the fixation portion, the mounting portion configured to hold a component within the recipient's body, the mounting portion comprising:
a collar configured to extend at least partially around the component; and
a reservoir configured to receive an adhesive material within a predetermined volume at least partially bounded by an inner surface of the collar and an outer surface of the component.

2. The apparatus of claim 1, wherein the collar is configured to allow the component to be adjustably moved within the collar along a longitudinal axis of the component.

3. The apparatus of claim 1, wherein the collar comprises a ring configured to extend around a substantially cylindrical portion of the outer surface of the component.

4. The apparatus of claim 1, wherein the inner surface of the collar comprises:
a substantially cylindrical first portion; and
a second portion that extends radially outwardly further than does the substantially cylindrical first portion, the volume at least partially bounded by the second portion of the inner surface and the outer surface of the component.

5. The apparatus of claim 1, wherein the reservoir comprises at least one concave channel extending along the inner surface of the collar, the channel bounded by the inner surface of the collar and the outer surface of the component.

6. The apparatus of claim 1, wherein the volume is selected to contain a predetermined amount of the adhesive material.

7. The apparatus of claim 1, wherein the reservoir comprises an inlet configured to receive the adhesive material with the component positioned within the collar and the fixation portion affixed to the bone surface.

8. (canceled)

9. (canceled)

10. The apparatus of claim 1, wherein the fixation portion comprises a first portion configured to be affixed to a first location and a second portion extending from the first portion towards a target portion of the recipient's body.

11. (canceled)

12. The apparatus of claim 1, wherein the component comprises a transducer.

13. An apparatus comprising:
a first component configured to be implanted within a recipient's body through a surgical hole; and
a second component configured to be implanted within the recipient's body through the surgical hole, the second component having a first portion configured to be affixed to the recipient's body and a second portion configured to be affixed to the first component, the second portion comprising a receptacle configured to hold a predetermined amount of adhesive between the first component and the receptacle.

14. The apparatus of claim 13, wherein the receptacle comprises an opening at an edge of the second portion.

15. The apparatus of claim 14, wherein the edge is configured to face the surgical hole upon implantation of the second component.

16. The apparatus of claim 13, wherein the first component is a microphone, an actuator, or an optical sensor and the second component is a fixation element.

17. The apparatus of claim 13, wherein the receptacle comprises a region between an inner surface of the second component and an outer surface of the first component.

18. The apparatus of claim 17, wherein one or both of the inner surface of the second component and the outer surface of the first component comprises a concave portion configured to receive and hold the adhesive.

19. The apparatus of claim 13, wherein the first component is configured to be in operational communication with a target portion of the recipient's body, the target portion spaced from the second component.

20. (canceled)

21. (canceled)

22. (canceled)

23. A method comprising:
affixing a fixation element to a location within a recipient's body, the fixation element configured to receive an assembly;
inserting the assembly into the fixation element such that a position of the assembly can be adjusted relative to the fixation element;
slidably adjusting the position of the assembly such that the assembly is at a location at which the assembly is configured to be operationally coupled to a target portion of the recipient's body; and
upon the assembly being at the location, filling a volume between the fixation element and the assembly with adhesive.

24. The method of claim 23, wherein the volume is between an inner surface of the fixation element and an outer surface of the assembly.

25. The method of claim 23, wherein the fixation element comprises a guide element indicative of the volume containing a predetermined amount of the adhesive, and said filling comprises stopping said filling of the volume with the adhesive when the adhesive reaches the guide element.

26. The method of claim 23, wherein the method further comprises attaching the assembly to the target portion of the recipient's body.

27. (canceled)

28. (canceled)

29. (canceled)
